(19) United States
(12) Patent Application Publication
(10) Pub. No.: US 2012/0052014 A1
(43) Pub. Date: Mar. 1, 2012

(54) FERRITIN BIOSENSOR AND METHODS OF USING THE SAME

(76) Inventors: Sufi Oasim Raja, West Bengal (IN); Rupak Kumar Chaudhuri, Kolkata (IN); Rajib De, West Bengal (IN); Anjan Kr. Dasgupta, Kolkata (IN)

(21) Appl. No.: 13/055,896

(22) PCT Filed: Aug. 2, 2010
(86) PCT No.: PCT/IB10/01900

§ 371 Date: Jan. 25, 2011

(30) Foreign Application Priority Data
May 11, 2010 (IN) 522/KOL/2010

(51) Int. Cl. A61K 49/08; G01N 33/20; B82Y 15/00

(52) U.S. Cl. 424/9.3; 436/84; 977/773; 977/930

(57) ABSTRACT

Disclosed herein are methods of diagnosing, monitoring and treating iron disorders using assays of serum ferritin. Some embodiments relate to detection and management of iron and iron-containing proteins in the body. For instance, the level of ferritin may be determined by contacting a sample with one or more magnetic nanoparticles; measuring an absorbance of the sample in the presence and in the absence of a magnetic field; and comparing the absorbance of the sample in the presence of the magnetic field to the absorbance of the sample in the absence of the magnetic field to determine the level of ferritin in the sample.

### TECHNICAL FIELD

The present technology relates generally to biosensors to detect analytes in a sample and to methods for using such biosensors.

### BACKGROUND

The following description is provided to assist the understanding of the reader. None of the information provided or references cited is admitted to be prior art to the present technology.

Free iron is toxic to cells as it acts as a catalyst in the formation of free radicals from reactive oxygen species. Within cells, iron may be stored safely in a protein complex as ferritin or hemosiderin. Ferritin is an iron storage protein in which iron is stored in an apoferritin core. Apoferritin binds to free ferrous iron, storing it in the ferric state. In this manner, ferritin stores iron in a soluble and non-toxic form, allowing its release in a controlled fashion in single cell and multi-celled organisms. In humans, ferritin acts as a buffer against iron deficiency and iron overload. Low levels of serum ferritin could indicate the first stage of iron depletion as found in various forms of anemia. Enhanced serum ferritin levels could indicate hemochromatosis, stress and/or inflammation. Iron overload is also observed in allogeneic hematopoietic cell transplantation.

Because serum iron and serum ferritin increase and decrease together, accurate measurements of the iron level in the body may be determined by measuring the serum ferritin. Thus, accurate measurement of the iron content of serum ferritin can improve the detection of the patient's iron status.

### SUMMARY

In one aspect, the disclosure provides a method for measuring the level of ferritin in a sample from a subject, the method comprising: contacting the sample with one or more magnetic nanoparticles; measuring an absorbance of the sample in the presence and in the absence of a magnetic field; and comparing the absorbance of the sample in the presence of the magnetic field to the absorbance of the sample in the absence of the magnetic field to determine the level of ferritin in the sample. In one embodiment, the sample is a body fluid sample. In one embodiment, the body fluid sample is a serum sample.

In one embodiment, the method further comprises comparing the measured level of ferritin in the sample to a reference level, wherein a difference in the level of ferritin in the sample compared to a reference level indicates a diagnosis of an iron-related disorder in the subject. In one embodiment, the method further comprises comparing the measured level of ferritin in the sample to a reference level, wherein a measured level of ferritin in the sample that is substantially the same as the reference level indicates the absence of an iron-related disorder in the subject.

In one embodiment, the one or more magnetic nanoparticles are superparamagnetic iron oxide nanoparticles (SPIONs). In one embodiment, the SPIONs are functionalized SPIONs. In one embodiment, the functionalized SPIONs are functionalized with citrate. In one embodiment, the SPIONs have a polydispersity from about 0.25 to about 0.29 In one embodiment, the SPIONs have a mean diameter from about 18 nm to about 22 nm. In one embodiment, the absorbance is measured at about 280 nm.

In one aspect, the disclosure provides an in vivo method for measuring the level of ferritin in a subject, the method comprising: administering to the subject an effective amount of one or more magnetic nanoparticles; measuring one or more physical characteristics of the subject in the presence of a magnetic field; and comparing the one or more physical characteristics in the presence of the magnetic field to a reference level to determine the level of ferritin in the subject. In one embodiment, the one or more physical characteristics include a relaxivity R2 measured by MRI. In one embodiment, the one or more physical characteristics are the optical properties of a body region. In one embodiment, the optical properties of a body region include the optical absorbance, emission, or scattering of the body region.

In another aspect, the disclosure provides a biosensor apparatus comprising: a sample chamber for receiving a sample; one or more magnetic nanoparticles in fluid communication with the sample chamber; a magnetic field generator capable of generating a magnetic field on the sample chamber; and an absorbance detector to measure the absorbance of the sample in the sample chamber. In one embodiment, the one or more magnetic nanoparticles are superparamagnetic iron oxide nanoparticles (SPIONs). In one embodiment, the magnetic field generator is magnet or an electromagnet. In one embodiment, the absorbance detector is a spectrophotometer.

In another aspect, the disclosure provides a kit for measuring the level of ferritin in a sample from a subject, the kit comprising: one or more magnetic nanoparticles; a magnet; and a standard curve for estimating ferritin concentration from based on a detected absorbance. In one embodiment, the kit further comprises an absorbance detector. In one embodiment, the absorbance detector is a spectrophotometer.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 depicts a block diagram of an illustrative embodiment of ferritin biosensor system.

FIG. 2 is an illustrative embodiment of a dynamic light scattering study showing the nanoparticle size.

FIG. 3A shows an illustrative embodiment of ZFC and FC magnetization curves taken at 100 Oe. FIG. 3B shows an illustrative embodiment of magnetization (M) versus applied magnetic field (H) of the sample taken at 5K, 100K, 200K and 300K up to 4 T. FIG. 3C shows an illustrative embodiment of magnetization re-plotted from FIG. 3B as a function of H/T.

FIG. 4 shows an illustrative model for iron-containing serum protein interaction with SPIONs (without and with magnetization) based on the observation of polydispersity index (PDI) measured using DLS.

FIG. 5 shows an illustrative embodiment of the ferritin level (X-axis) dependence of percentage change in magnetically induced capture of protein as measured by a fractional change in absorbance at 280 nm (Y-axis). Open circles are the ferritin level below the critical point (Fc) and squares are the ferritin level beyond that critical point.

FIG. 6 is an illustrative embodiment of a magnetoscopic device for measuring serum ferritin in vivo.

FIG. 7 shows an illustrative embodiment of the peroxidase activity of SPIONs (Michealis-Menten Kinetics) measured using TMB as substrate.

### DETAILED DESCRIPTION

In the following detailed description, reference may be made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here. In the description that follows, a number of terms are used extensively. The terms described below are more fully understood by reference to the specification as a whole. Units, prefixes, and symbols may be denoted in their accepted SI form.

The terms “a” and “an” as used herein mean “one or more” unless the singular is expressly specified. Thus, for example, reference to a “nanoparticle” includes a mixture of two or more such nanoparticles, as well as a single nanoparticle.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the “administration” of an agent to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The terms “assessing” and “evaluating” are used interchangeably to refer to any form of measurement, and include determining if a characteristic, trait, or feature is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample; for example, the sample may be frozen at about −20° C. to about −70° C. An “acellular” body fluid contains no intact cells. Examples of acellular fluids include plasma or serum, or body fluids from which cells have been removed.

The term “comparable” or “corresponding” in the context of comparing two or more samples means that the same type of sample (e.g., serum) is used in the comparison. For example, a level of ferritin in a serum sample can be compared to a level of ferritin in another serum sample. In some embodiments, comparable samples may be obtained from the same individual at different times. In other embodiments, comparable samples may be obtained from different subjects (e.g., a patient and a healthy individual). In general, comparable samples are normalized by a common factor. For example, acellular body fluid samples are typically normalized by volume body fluid and cell-containing samples are normalized by protein content or cell count.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical art for a particular disease or disorder, e.g., an iron-related disorder.

As used herein, the phrase “difference of the level” refers to differences in the quantity of a particular marker, such as serum ferritin, in a sample as compared to a control or reference level. For example, the quantity of particular protein may be present at an elevated amount or at a decreased amount in samples of patients with a iron-related disorder compared to a reference level. In one embodiment, a “difference of a level” may be a difference between the level of ferritin present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the levels of ferritin present in a sample as compared to a control. For example, a difference may be statistically significant if the measured level of ferritin falls outside of about 1.0 standard deviations, about 1.5 standard deviations, about 2.0 standard deviations, or about 2.5 stand and deviations of the mean of any control or reference group.

As used herein, the term “effective amount” refers to a quantity sufficient to achieve a desired therapeutic, prophylactic, or diagnostic effect. For therapeutic and prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the term “nanoparticle” refers to any material having dimensions in the 1-1,000 nm range. In some embodiments, nanoparticles have dimensions in the 2-200 nm range, in the 2-150 nm range, or in the 2-100 nm range. Nanoparticles used in the present compositions and methods include, but are not limited to, nanoscale materials such as a superparamagnetic nanoparticle.

As used herein, the term “superparamagentic iron oxide nanoparticle” or “SPION” refers to superparamagnetic iron oxide crystalline structures that have the general formula [Fe₂⁺O₃]ₓ[Fe₂⁺O₃(M²⁺O)]₁₋ₓ where 1≧x≧0, M²⁺ may be a divalent metal ion such as iron, manganese, nickel, cobalt, magnesium, copper, or a combination thereof. For example, when the metal ion (M²⁺) is ferrous ion (Fe²⁺) and x=0, the SPION is magnetite (Fe₃O₄). In general, superparamagnetism occurs when crystal-containing regions of unpaired spins are sufficiently large that they can be regarded as thermodynamically independent, single domain particles called magnetic domains. These magnetic domains display a net magnetic dipole that is larger than the sum of its individual unpaired electrons. In the absence of an applied magnetic field, all the magnetic domains are randomly oriented with no net magnetization. Application of an external magnetic field causes the dipole moments of all magnetic domains to reorient resulting in a net magnetic moment.

As used herein, “plasma” refers to acellular fluid found in blood. Plasma may be obtained from blood by removing whole cellular material from blood by methods known in the art (e.g., centrifugation, filtration, and the like). As used herein, “peripheral blood plasma” refers to plasma obtained from peripheral blood samples.

As used herein the term “polydispersity” generally refers to variability of component size within a given sample. For example, the polydispersity of nanoparticles may be shown by transmission electron microscopy (TEM) of the imaging of the agent. The disparity values for the nanoparticle cores reported herein are the standard deviation from a statistical analysis of the iron oxide cores by TEM image analysis. The polydispersity of the disclosed SPION may also be measured using dynamic light scattering to determine the hydrodynamic diameter (D_{H}).

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for an iron-related disorder if, after receiving a therapeutic amount of the active agents according to the methods described herein, the subject shows observable and/or measurable reduction in or absence of one or more signs and symptoms of an iron-related disorder, such as a normal level of serum ferritin. It is also to be appreciated that the various modes of treatment or prevention of medical conditions as described are intended to mean “substantial”, which includes total but also less than total treatment or prevention, and wherein some biologically or medically relevant result is achieved.

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “drug,” “compound,” “active agent,” “actives,” “pharmaceutical composition,” “pharmaceutical formulation,” and “pharmacologically active agent” are used interchangeably herein to refer to any compound, complex, or composition that is suitable for administration and that has a beneficial biological effect, suitably a therapeutic effect in the treatment of an iron-related disorder, although the effect may also be prophylactic in nature. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “drug” are used, then, or when a particular active agent is specifically identified, it is to be understood to include the active agent per se as well as pharmaceutically acceptable, pharmacologically active salts, esters, amides, prodrugs, conjugates, metabolites, analogs, etc.

As used herein, the term “reference level” refers to a level of a substance which may be of interest for comparative purposes. In one embodiment, a reference level may be the level of ferritin expressed as an average of the level of ferritin from samples taken from a control population of healthy (disease-free) subjects. In another embodiment, the reference level may be the level in the same subject at a different time, e.g., before the present assay, such as the level determined prior to the subject developing the disease or prior to initiating therapy. In general, samples are normalized by a common factor. For example, acellular body fluid samples are normalized by volume body fluid and cell-containing samples are normalized by protein content or cell count.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascites fluid, or whole blood, and including biopsy samples of body tissue. A sample may be obtained from any subject, e.g., a subject/patient having or suspected to have an iron-related disorder. A sample may also be obtained from a subject/patient for the purposes of a routine screening procedure, even if that subject is not suspected to have an iron-related disorder.

As used herein, “serum” includes the fraction of plasma obtained after plasma or blood is permitted to clot and the clotted fraction is removed.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a disease or disorder.

The phrase “substantially the same as” in reference to a comparison of one value to another value for the purposes of clinical management of a disease or disorder means that the values are statistically not different. Differences between the values can vary, for example, one value may be within 20%, within 10%, or within 5% of the other value.

The terms “optional” and “optionally” mean that the subsequently described circumstance may or may not occur, so that the description includes instances where the circumstance occurs and instances where it does not.

### Overview

Disclosed herein are methods for detecting the presence or absence of iron-related disorders in subjects based, at least in part, on results of testing methods of the present technology on a sample. Iron-related disorders include, for example, hemochromatosis, thalassemia major, siderochrestic anemia, Blackfan-Diamond anemia, aplastic anemia, sickle cell anemia, hemolytic anemias and hemosiderosis. Further disclosed herein are methods for monitoring the status of subjects diagnosed with iron-related disorders based at least partially on results of tests on a sample. The test samples disclosed herein are represented by, but not limited in anyway to, sputum, blood (or a fraction of blood such as plasma, serum, or particular cell fractions), lymph, mucus, tears, saliva, urine, semen, ascites fluid, whole blood, and biopsy samples of body tissue. This disclosure is drawn, inter alia, to methods of diagnosing, monitoring and treating iron disorders using assays of serum ferritin. Some embodiments relate to detection and management of iron and iron-containing proteins in the body and measuring their interactions from outside the body.

Nearly 99% of body iron in humans and many mammals is heme iron. Heme iron is typically complexed with protein, forming hemoproteins. The remaining 1% of body iron includes ferritin, transferrin, hemosiderin, and iron chelated with albumin and/or citrate. The stored iron in ferritin is non-heme in nature and shows a crystal arrangement like ferrihydrite mineral with a remarkable magnetic moment (for example, commercial horse spleen ferritin showed a magnetic moment of 1.8 emu/gm with a characteristic blocking temperature of approximately 15K as determined by SQUID analysis). Various embodiments of the present methods relate to detecting a magnetic interaction between ferritin and a magnetic nanoparticle, such as a super paramagnetic iron oxide nanoparticle (SPION). Without wishing to be limited by theory, the present systems and methods are based on a magnetic interaction between magnetic nanoparticles and body fluids which have a ferromagnetic component (e.g., ferritin). In some embodiments, the magnetic response is linear below a ferritin concentration of about <1000 ng/ml.

### Magnetic Nanoparticles

Any nanoparticle which meets the size and magnetic criteria can be used as a component of a biosensor in the systems and methods disclosed herein. In one embodiment, the nanoparticles are magnetic nanoparticles. In one embodiment, the nanoparticles are superparamagnetic. Super paramagnetic particles are crystalline particles of a magnetic medium that are so small that their magnetization can randomly flip direction under the influence of temperature. Nanoscale superparamagnetic particles are also known as super paramagnetic nanoparticles (SPN). Super paramagnetic nanoparticles may include, for example, iron oxide or nickel ferrite.

Nanoscale iron oxide super paramagnetic particles as also known as super paramagnetic iron oxide nanoparticles (SPIONs). In one embodiment, the SPION may be a colloidal SPION. A colloidal SPION is a mixture of SPIONs in a continuous liquid phase. In order to maintain the continuous liquid phase, the nonoparticles have a surface potential. For example, if the surface potential is on the order of 20 mV, the electric repulsion is strong enough to maintain the colloidal state. In SPIONs, apart from the hydrophobic interaction between different colloidal particles, the magnetic interaction between them also plays a role in maintaining the colloidal stability. A surface potential can originate from surface functionalization (described below).

In some embodiments, the SPIONs may have different surface functionalization. Generally, bare nanoparticles are toxic to cells, so the surface of nanoparticles may be functionalized with agents which are nontoxic and biocompatible. Example agents include, but are not limited to, citrate, poly-ethylene glycol (PEG), poly-vinyl alcohol (PVA), dextran. In two embodiments, the SPIONs are functionalized by citrate or dextran. Additional examples of surface functionalization are shown in Table 1.

TABLE 1
Different classes of surface functionalization for SPIONs

| | | |
|---|---|---|
| Natural Polymers | Dextran | Optimizes polar interaction with SPION |
| | Starch | Good for MRI and drug delivery |
| | Gelatin | Used as a biocompatible gelling agent |
| | Chtitosan | Non-Viral gene delivery system |
| Synthetic Polymer | PEG | Improves blood circulation |
| | PVA | Prevents agglomeration |
| | PLA | Improves biodegradability |
| | Alginate | Improves Stability |
| | PMMA | Used as thermo sensitive drug delivery |
| | | system |
| | PAA | Improves Stability as well as |
| | | bio-conjugation |

The SPIONs for use in the present methods can be prepared and stored by any method known to those of skill in the art. Commercially prepared SPIONs can also be used. An illustrative method for preparing SPIONs is described in Example 1.

### In Vitro Methods

In one aspect, the disclosure provides an in vitro method for measuring the level of ferritin in a sample from a subject by contacting the sample with one or more magnetic nanoparticles; The contact eventually facilitates the interaction of two classes of magnetic objects, namely the ferritin and the SPIONS. The interaction can be studied by comparing the SPION bound ferritin in presence of magnetic field. As the ferritin SPION complex forms a cluster that can be separated out by low speed centrifugation (e.g., at 2000 rpm for 10 min) the extent of the binding can be quantified by measurement of the relative levels of either the nanoparticles or the protein. In one embodiment, the protein amount can be measured by detecting tryptophan absorbance at 280 nm. In another embodiment, the relative amount of magnetic nanoparticles can be measured using the peroxidase activity of SPIONs and ferritin. (See Victor C. Yang, et al; Biomaterials (2009), 30, 4716-4722). The calibration with different known levels of pure ferritin provides a quantitative measure of the ferritin level in a test sample (e.g. serum). Comparisons may be made between the measured level of ferritin in the subject to a reference level to diagnose an iron-related disorder. The disclosure further relates to ferritin biosensor apparatuses and systems for measuring serum ferritin in biological samples (e.g. serum).

Test samples of acellular body fluid or cell-containing samples may be obtained from an individual or patient. Methods of obtaining test samples are well-known to those of skill in the art and include, but are not limited to, aspirations or drawing of blood or other fluids. Samples may include, but are not limited to, whole blood, serum, plasma, saliva, cerebrospinal fluid (CSF), pericardial fluid, pleural fluid, urine, and eye fluid.

In embodiments in which the ferritin level will be determined using an acellular body fluid, the test sample may be a cell-containing liquid or an acellular body fluid (e.g., plasma or serum). In some embodiments in which the test sample contains cells, the cells may be removed from the liquid portion of the sample by methods known in the art (e.g., centrifugation) to yield acellular body fluid for the ferritin level measurement. In suitable embodiments, serum or plasma are used as the acellular body fluid sample. Plasma and serum can be prepared from whole blood using suitable methods well-known in the art. In these embodiments, data may be normalized by volume of acellular body fluid.

Variability in sample preparation of cell-containing samples can be corrected by normalizing the data by, for example, protein content or cell number. In certain embodiments, ferritin level in the sample may be normalized relative to the total protein content. Total protein content in the sample can be determined using standard procedures, including, without limitation, Bradford assay and the Lowry method. In other embodiments, ferritin level in the sample may be normalized relative to cell number.

In certain embodiments, the level of ferritin in a test sample from a patient is used in the diagnosis of disease, such as an iron disorder. An iron disorder is any condition in which there is too much or too little iron for the body to function normally. Individuals with iron disorders can have many vague symptoms or health issues including any one or combination of the following: fatigue, joint pain, bone or joint disease (osteoarthritis, osteoporosis), shortness of breath, irregular heart beat, liver trouble, diabetes, infertility, impotence, depression, mood or mental disorders, poor cognitive skills or neurodegenerative diseases. Iron-related diseases include, for example, hemochromatosis, thalassemia major, sideroachrestic anemia, Blackfan-Diamond anemia, aplastic anemia, sickle cell anemia, hemolytic anemias and hemosiderosis.

Hemochromatosis (HH) is a disease that results from excessive amounts of iron in the body (iron overload). Hereditary (genetic) hemochromatosis (HHC) an inherited disorder of abnormal iron metabolism. Individuals with hereditary hemochromatosis absorb too much dietary iron. Once absorbed, the body does not have an efficient way of excreting iron excesses. Over time, these excesses build to a condition of iron overload, which is a toxic to cells. Glands and organs, including the liver, heart, pituitary, thyroid, pancreas, synovium (joints) and bone marrow burdened with excess iron cannot function properly. Symptoms develop and disease progresses. There are several types of genetic hemochromatosis. These include: Type I or Classic (HHC); Type II a, b or Juvenile (JHC); Type III or Transferrin Receptor Mutation; and Type IV or Ferroportin Mutation.

In some embodiments, the level of ferritin in a test sample is used to diagnose a disease. The level of ferritin may be compared to a reference value to determine if the level of ferritin is elevated or reduced relative to the reference value. Typically, the reference value is the level of ferritin measured in a comparable sample from one or more healthy individuals. An increase or decrease in the level of ferritin may be used in conjunction with clinical factors to diagnose a disease.

Association between a pathological state (e.g., an iron disorder) and the aberration of the level of ferritin can be readily determined by comparative analysis in a normal population and an abnormal or affected population. Thus, for example, one can study the level of ferritin in both a normal population and a population affected with a particular pathological state. The study results can be compared and analyzed by statistical means. Any detected statistically significant difference in the two populations would indicate an association. For example, if the serum ferritin is statistically significantly higher in the affected population than in the normal population, then it can be reasonably concluded that higher serum ferritin is associated with the pathological state.

Statistical methods can be used to set thresholds for determining when the ferritin level in a subject can be considered to be different than or similar to a reference level. In addition, statistics can be used to determine the validity of the difference or similarity observed between a patient's ferritin level and the reference level. Useful statistical analysis methods are described in L. D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between groups deemed significant if the p value is less than or equal to 0.05. As used herein a “confidence interval” or “CI” refers to a measure of the precision of an estimated or calculated value. The interval represents the range of values, consistent with the data that is believed to encompass the “true” value with high probability (usually 95%). The confidence interval is expressed in the same units as the estimate or calculated value. Wider intervals indicate lower precision; narrow intervals indicate greater precision. Suitable confidence intervals are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Suitable p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistics for Research, John Wiley & Sons, New York, 1983.

Once an association is established between an aberrant ferritin level and a pathological state, then the particular physiological state can be diagnosed or detected by determining whether a patient has the particular aberration, i.e. elevated or reduced ferritin levels.

The term “elevated levels” or “higher levels” as used herein refers to levels of ferritin that are higher than what would normally be observed in a comparable sample from control or normal subjects (i.e., a reference value). In some embodiments, “control levels” (i.e., normal levels) refer to a range of ferritin levels that would be normally be expected to be observed in a mammal that does not have an iron disorder. A control level may be used as a reference level for comparative purposes. “Elevated levels” refer to ferritin levels that are above the range of control levels. The ranges accepted as “elevated levels” or “control levels” are dependent on a number of factors. For example, one laboratory may routinely determine the level of ferritin in a sample that are different than the level of ferritin obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated values”. For example, a series of samples from control subjects and subjects diagnosed with iron disorders can be used to establish ranges that are “normal” or “control” levels and ranges that are “elevated” or “higher” than the control range.

Similarly, “reduced levels” or “lower levels” as used herein refer to levels of ferritin that are lower than what would normally be observed in a comparable sample from control or normal subjects (i.e., a reference value). In some embodiments, “control levels” (i.e. normal levels) refer to a range of ferritin levels that would be normally be expected to be observed in a mammal that does not have a iron disorder and “reduced levels” refer to ferritin levels that are below the range of such control levels.

### Ferritin Biosensors

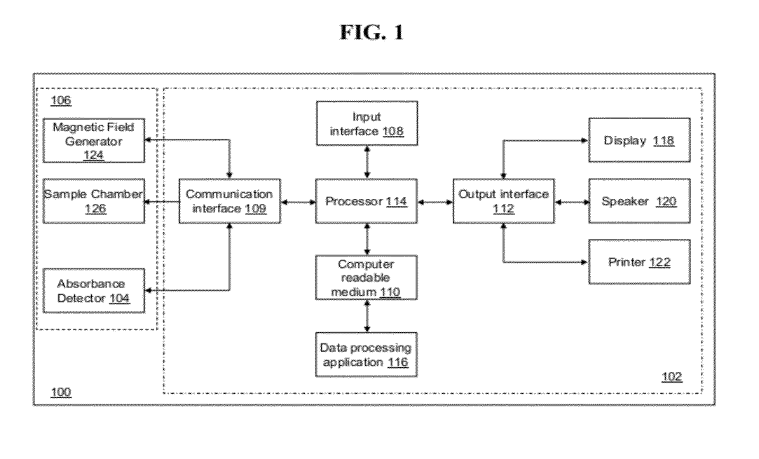

In one aspect, the present disclosure provides a biosensor apparatus for detecting or measuring the level of ferritin in a sample. With reference to FIG. 1, a block diagram of a system for measuring the level of ferritin is shown in accordance with an illustrative embodiment. Ferritin biosensor 100 may include one or more of a computing system 102, a absorbance detector 104, and a sample analysis instrument 106. Different and additional components may be incorporated into ferritin biosensor 100. Computing system 102 may include one or more of an input interface 108, a communication interface 109, a computer-readable medium 110, an output interface 112, a processor 114, a data processing application 116, a display 118, a speaker 120, and a printer 122. Different and additional components may be incorporated into computing system 102.

Input interface 108 provides an interface for receiving information from the user for entry into computing system 102 as known to those skilled in the art. Input interface 108 may use various input technologies including, but not limited to, a keyboard, a pen and touch screen, a mouse, a track ball, a touch screen, a keypad, one or more buttons, etc. to allow the user to enter information into computing system 102 or to make selections presented in a user interface displayed on display 118. The same interface may support both input interface 108 and output interface 112. For example, a touch screen both allows user input and presents output to the user. Computing system 102 may have one or more input interfaces that use the same or a different input interface technology.

Communication interface 109 provides an interface for receiving and transmitting data between devices using various protocols, transmission technologies, and media as known to those skilled in the art. Communication interface 109 may support communication using various transmission media that may be wired or wireless. Computing system 102 may have one or more communication interfaces that use the same or a different communication interface technology. Data and messages may be transferred between computing system 102, absorbance detector 104, and/or sample analysis instrument 106 using communication interface 109.

Computer-readable medium 110 is an electronic holding place or storage for information so that the information can be accessed by processor 114 as known to those skilled in the art. Computer-readable medium 110 can include, but is not limited to, any type of random access memory (RAM), any type of read only memory (ROM), any type of flash memory, etc. such as magnetic storage devices (e.g., hard disk, floppy disk, magnetic strips, etc.), optical disks (e.g., CD, DVD, etc.), smart cards, flash memory devices, etc. Computing system 102 may have one or more computer-readable media that use the same or a different memory media technology. Computing system 102 also may have one or more drives that support the loading of a memory media such as a CD or DVD. Computer-readable medium 110 may provide the electronic storage medium for fluorescence detector 104 and/or sample analysis instrument 106. Computer-readable medium 110 further may be accessible to computing system 102 through communication interface 109.

Output interface 112 provides an interface for outputting information for review by a user of computing system 102. For example, output interface 112 may include an interface to display 118, speaker 120, printer 122, etc. Display 118 may be a thin film transistor display, a light emitting diode display, a liquid crystal display, or any of a variety of different displays known to those skilled in the art. Speaker 120 may be any of a variety of speakers as known to those skilled in the art. Printer 122 may be any of a variety of printers as known to those skilled in the art. Computing system 102 may have one or more output interfaces that use the same or a different interface technology. Display 118, speaker 120, and/or printer 122 further may be accessible to computing system 102 through communication interface 109.

Processor 114 executes instructions as known to those skilled in the art. The instructions may be carried out by a special purpose computer, logic circuits, or hardware circuits. Thus, processor 114 may be implemented in hardware, firmware, or any combination of these methods and/or in combination with software. The term “execution” is the process of running an application or the carrying out of the operation called for by an instruction. The instructions may be written using one or more programming language, scripting language, assembly language, etc. Processor 114 executes an instruction, meaning that it performs/controls the operations called for by that instruction. Processor 114 operably couples with input interface 108, with communication interface 109, with computer-readable medium 110, and with output interface 112, to receive, to send, and to process information. Processor 114 may retrieve a set of instructions from a permanent memory device and copy the instructions in an executable form to a temporary memory device that is generally some form of RAM. Computing system 102 may include a plurality of processors that use the same or a different processing technology.

Data processing application 116 performs operations associated with processing data for a sample gathered using one or more electronic devices that continuously, periodically, and/or upon request monitor, sense, measure, etc. the physical and/or chemical characteristics of the sample. The operations may be implemented using hardware, firmware, software, or any combination of these methods. With reference to the illustrative embodiment of FIG. 1, data processing application 116 is implemented in software (comprised of computer-readable and/or computer-executable instructions) stored in computer-readable medium 110 and accessible by processor 114 for execution of the instructions that embody the operations of data processing application 116. Data processing application 116 may be written using one or more programming languages, assembly languages, scripting languages, etc.

Absorbance detector 104 may include an absorbance detection system such as a spectrophotometer, etc. Absorbance detector 104 generates data related to a sample, such as the A₂₈₀ from the sample. The source of and the dimensionality of the data is not intended to be limiting. Computing system 102 may be separate from or integrated with absorbance detector 104 to control the operation of absorbance detector 104.

Sample analysis instrument 106 may include magnetic field generator 124 capable of generating a magnetic field upon the sample chamber 126. Different and additional components may be incorporated into sample analysis instrument 106. Magnetic field generator 124 produces magnetism to detect an interaction between one or more magnetic nanoparticles and ferritin in the sample.

In one embodiment, the biosensor system 100 is a handheld device that includes a spectrophotometer and a microfluidic chip (not shown). The microfluidic chip may be an integrated chip that may include reservoirs, reaction chambers, and plumbing for the sample and SPIONs. The microfluidic chip may be a microelectromechanical system (MEMS) chip. A MEMS chip typically includes both electrical and mechanical components and may include, for example, a microprocessor and one or more mechanical pumps.

### In Vivo Methods

In one aspect, the disclosure provides an in vivo method for measuring the level of ferritin in a subject by administering to the subject an effective amount of one or more magnetic nanoparticles; measuring one or more physical characteristics (e.g., absorbance or magnetic response) of the subject in the presence and in the absence of a magnetic field; and comparing the one or more physical characteristics in the presence of the magnetic field to the response of the subject in the absence of the magnetic field to determine the level of ferritin in the subject. In some embodiments, magnetic resonance imaging or optical detection may be used to detect a magnetic interaction between the one or more magnetic nanoparticles and the serum ferritin.

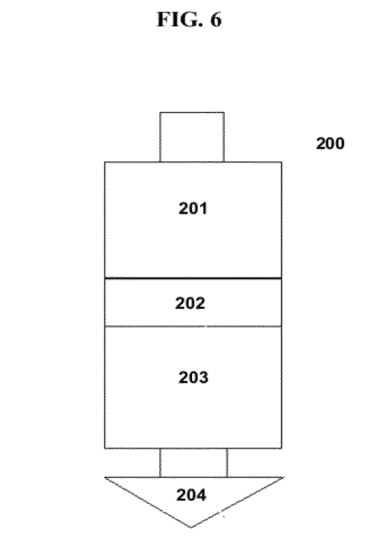

In some embodiments, the magnetic properties of SPIONs can be modulated by applying an external magnetic field to the body of a subject or a region of the body of the subject. The SPIONs in turn can interact with ferritin. In one embodiment, the ferritin level is measured optically using an endoscopic device 200, which is illustrated in FIG. 6. The optical device 200 is based on a miniaturized form of a SPR-coupled endoscopic device, in which an electromagnet surrounds the SPIONs (e.g., dextran coated SPIONs). The device 200 contains a laser source and a photodetector 201. The SPION chamber is separated from the SPR device by a thin gold plate 202 (which is the SPR source). The binding of SPION with ferritin in the endoscopic device would cause alteration of local dielectric behavior. The system may be coupled to a programmable valve 204 that opens whenever the ferritin level is high. This may in turn release a drug, such as an iron chelating agent.

In another embodiment, the magnetic interaction of SPIONs and ferritin can be detected by modulation of an external magnetic field to the body of a subject or a region of the body of the subject. It is already reported that the ferritin is a poor MRI contrast agent with very low transverse relaxivity. However, in aggregated form, ferritin acts as a good MRI contrast agent and its relaxivity linearly increases (up to 10 mM⁻¹S⁻¹) with external field strength in the range of 1-10 T. In neurodegenerative diseases, ferritin accumulates in the different parts of the brain and remains in an aggregated form, which has been used as intrinsic MRI contrast agent for imaging the different parts of the brain.

Without wishing to be limited by theory, SPIONs can be used to increase the relaxivity of monomeric ferritin and the relaxivity can be detected. In an illustrative embodiment, the surface of the SPIONs can be coated with dextran to make the SPIONs more biocompatible and to increase the circulation time. Next, an in vitro R2 measurements of mixture of blood and SPIONs is used to obtain a standard curve correlating the R2 value and serum ferritin level. The controls will be pure dextran-coated SPIONs and blood containing different concentrations of ferritin. It is predicted that R2 will increase with an increase in serum ferritin level, because R2 increases with an increase in effective magnetic moment. As such, the magnetic moment increases with an increase in ferritin concentration.

In an illustrative embodiment, the in vivo MRI analysis can be performed on subjects using an 11.7 T horizontal magnet. It is reported (according to MICAD, Molecular Imaging and Contrast Agent Database) that after intravenous administration of dextran-coated SPIONs at 5 minutes, the concentration of SPIONs is at maximum. If the site of administration is targeted for imaging, the R2 value of the SPIONs conjugated ferritin in the blood can be determined. The R2 value can be used to find the serum ferritin level based on the standard curve of ferritin versus R2.

### EXAMPLES

The present technology, thus generally described, will be understood more readily by reference to the following examples, which are provided by way of illustration and are not intended to be limiting.

### Example 1

### SPIONs as a Ferritin Biosensor

### Materials and Methods

Synthesis of Iron Oxide nanoparticles. Magnetic iron oxide nanoparticles were prepared by co-precipitating 2 g ferrous chloride and 4 g ferric chloride (solubilized in 50 ml 2(N) HCl) by 150 ml 1.5(N) sodium hydroxide upon constant stirring at room temperature. The precipitate was washed well with milli-Q water and 20 ml citrate buffer (1.6 μcitric acid and 0.8 g tri-sodium citrate) was added to collect the stabilized nanoform in solution at a pH around 6.3. All these steps were performed in presence of a strong bar magnet to facilitate the process.

Preparation of blood serum. Blood samples were collected from individuals after obtaining informed consent patients admitted to Institute for Hematology and Transfusion Medicine (IHTM), Kolkata with normal and elevated level of serum ferritin. The study protocol was approved by the Institutional Ethical Committee of IHTM.

Interaction of Nanoparticles with human blood serum. Serum samples were centrifuged at 2000 rpm for 10 min and the supernatant was taken as working serum solution. In a 0.5 ml centrifuge tube, 10 μl nanoparticle solutions were added to 100 μl of serum. They were incubated both in absence and in presence of a strong bar magnet. After incubation, the supernatant and the pellet for both the sets were tested spectrophotometrically (using Perkin Elmer Lamda35 UV-Vis spectrophotometer). Absorbance was measured at 280 nm to measure the protein concentration in different samples. The difference in absorbance in presence and absence of magnetic field was calculated for the measurement of percent decrease of this absorbance with respect to initial absorbance at the said wavelength.

Characterization of SPIONs. Nanoparticle size was determined by Photon Correlation Spectroscopy (PCS) using the Nano-ZS (Malvern) instrument equipped with a 4 mW He—Ne Laser (λ=632 nm). The magnetic properties were characterized by Superconducting Quantum Interference Device (SQUID) using MPMS-7 (Quantum Design).

### Characterization of SPIONs

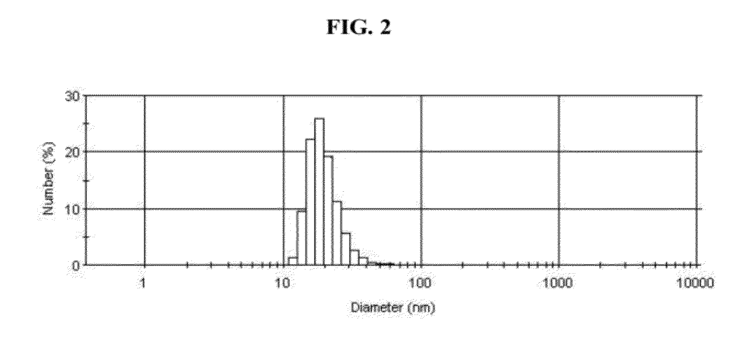

The particle size distribution by dynamic light scattering of the super paramagnetic iron oxide nanoparticle used for the study is shown in FIG. 2. FIG. 2 shows the plasmon behavior of the citrate-capped nanoparticle. The synthesized nanoparticles (synthesized by the route of synthesis shown above) had low polydispersity ˜0.27 and number distribution ˜20 nm.

Variation of magnetization (M) versus applied magnetic field (H) of the sample was measured at 5K, 100K, 200K and 300K up to 4 T and the measurement was performed in Superconducting Quantum Interference Device (SQUID). The measurements using SQUID are based on the principle of Brownian motion. Particles, emulsions and molecules in suspension undergo Brownian motion. This is the motion induced by the bombardment by solvent molecules that themselves are moving due to their thermal energy. If the particles or molecules are illuminated with a laser, the intensity of the scattered light fluctuates at a rate that is dependent upon the size of the particles as smaller particles are “kicked” further by the solvent molecules and move more rapidly. Analysis of these intensity fluctuations yields the profile of the autocorrelation function. The decay of the autocorrelation function is exponential in nature, with the exponent proportional to the diffusion coefficient of the particle, which in turn in dependent on the particle size.

The calculated value of the diameter (from blocking temperature) of the synthesized nanoparticles was ˜16 nm. The calculated value of the diameter of the iron oxide nanoparticle was determined using the equation TB=K*V/25 kB, where the magnetocrystalline anisotropy constant (K) of iron oxide nanoparticle varies from 1.4-5*10⁵ J/m³, TB is the blocking temperature (80K), and kB is the Boltzman constant.

The calculated value of the synthesized nanoparticles was is in good agreement with the experimentally found diameter (˜20 nm) through dynamic light scattering (DLS) study. DLS is sometimes referred to as Photon Correlation Spectroscopy (PCS) or Quasi-Elastic Light Scattering (QELS), is a non-invasive, technique for measuring the size of molecules and particles typically in the submicron region, and with the latest technology lower than 1 nm.

The synthesized nanoparticles (ferro-fluids) had low polydispersity ˜0.27 and number distribution ˜20 nm as explained above. There was a small population of large size particles, detectable only in the intensity distribution. The low polydispersity value implied pre-dominance of a single population.

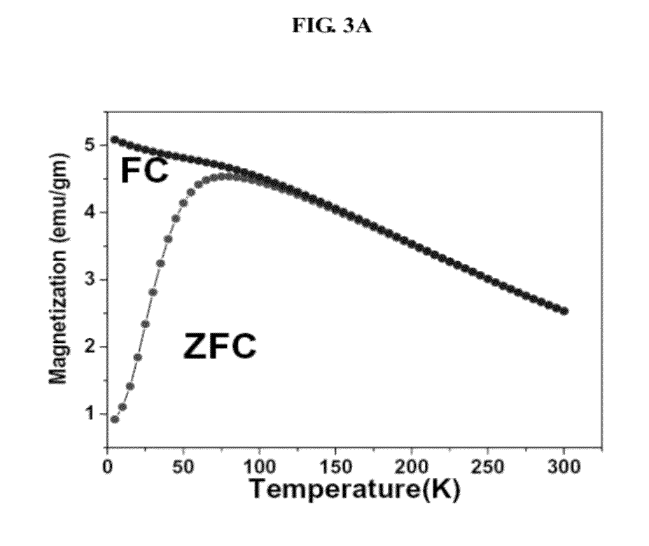

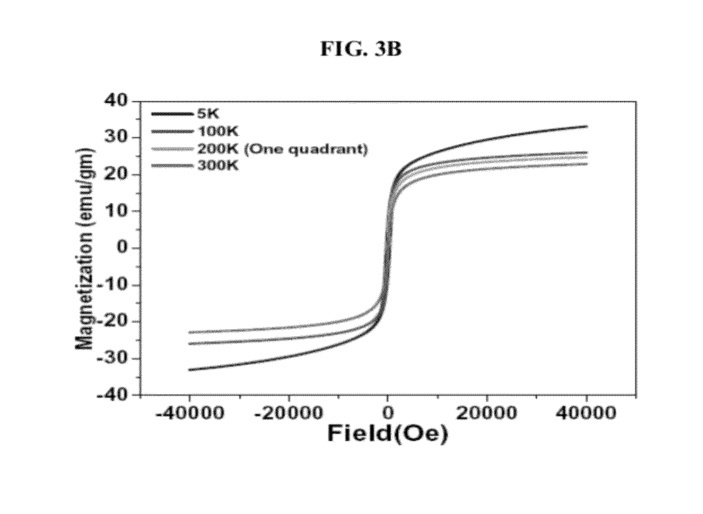

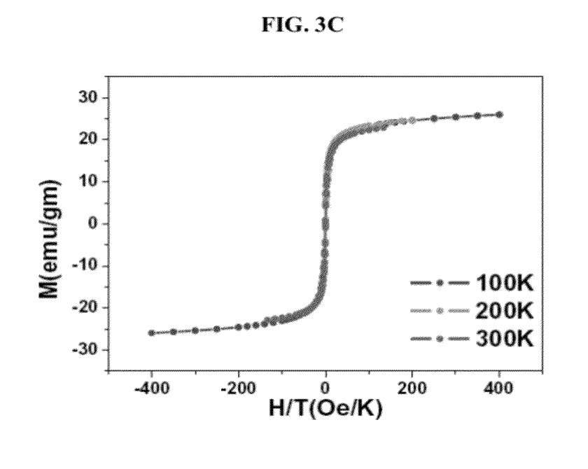

The existence of super-paramagnetic behavior of such particles was studied using ZFC and FC magnetization curves taken at 100 Oe. The distinct bifurcation of the ZFC and FC was observed around 80 K (FIG. 3A). The bifurcation indicates blocking temperature T_{B} as indicated by an arrow in the figure. The ZFC magnetization increases as a function of temperature for temperature 5K<T<80 K, indicating that there are blocked moments which start to contribute to the magnetization when the temperature was increased. In the same temperature region the FC magnetization decreases as the temperature increases, since the frozen moments start randomizing due to thermal energy. Beyond the blocking temperature both the ZFC and FC curve coincide and the magnetization decreases as the temperature increases due to randomization of the moments due to increase in the thermal energy upon increasing in temperature. This behavior was typical of superparamagnetic nanoparticle and was shown in FIG. 3A. FIG. 3B shows variation of magnetization (M) versus applied magnetic field (H) of the sample taken at 5K, 100K, 200K and 300K up to 4 T. No hysteresis was found for 100K, 200K and 300K data (above temperature T_{B}) and The M-H curve shows S-shaped behavior with saturation similar to a superparamagnetic behavior. No saturation was seen in the M-H curve taken at 5 K up to 4 T. To verify that the Fe₃O₄ particles are ideal superparamagnetic, one should observe that the magnetization isotherms should scale with H/T apart from no hysteresis. The magnetization from FIG. 3B was re-plotted in FIG. 3C as a function of H/T and it was observed the that the magnetization scales with H/T and all the three curves for temperature above T_{B} (100K, 200K and 300K) collapses on one single master curve as predicted for superparamagnetic nanoparticles, indicating that each of the particles are single domain.

### Assay of Serum Ferritin

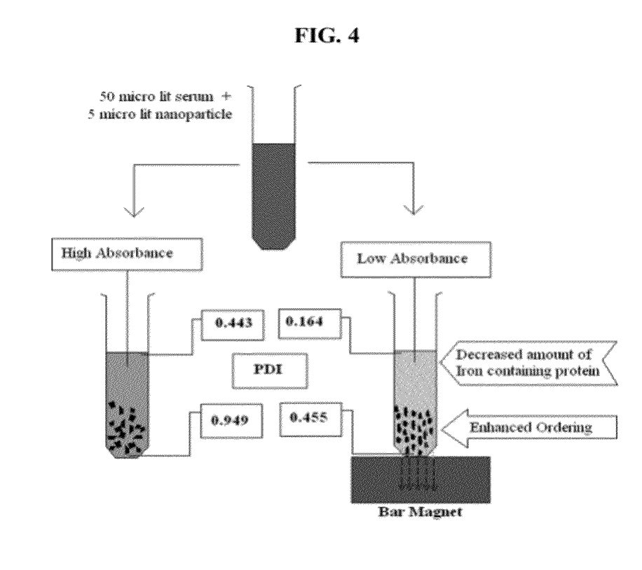

The next experimental work involved interaction of the nanoparticle synthesized with serum containing various iron containing proteins of which ferritin was a member. The interaction was studied by incubating the probe iron oxide nanoparticle with the serum in presence and absence of a weak magnet. It was observed that the polydispersity index (PDI), which is a strong indicator for species heterogeneity, was quite high for both the supernatant (0.443) and pellet (0.949) in the un-magnetized sample (see FIG. 4). However, this can be explained by the huge variety of proteins present in the serum and the diverse potency of the iron containing serum proteins to form micro-cluster with the magnetic nanoparticles respectively. But, these PDI values decrease significantly upon magnetization—the supernatant and pellet show the value 0.164 and 0.455 respectively, which was a two third reduction of the former value and a more than half for the later. Firstly, in case of the un-magnetized sample there are some un-reacted iron-containing proteins left in the supernatant. But, in presence of the strong magnetic field almost all the iron-containing protein population was pooled down via complexation, thus reducing the species heterogeneity in the supernatant. Consequently, the PDI value decreases significantly. Secondly, the decrease of PDI in re-suspended pellet for the magnetized sample was explained by the induced ordering of the magnetic clusters. Although there was more iron-containing protein in the pellet for magnetized sample, the protein-magnetic nanoparticle clusters are more aligned with each other due to the directionality of magnetic lines of forces. One point is that in all the cases (of different serum samples) there was a decrease in protein absorbance when serum was incubated with synthesized nanoparticle in presence of a weak magnet. This decrease was evidently due to higher interaction of the iron containing serum proteins with the iron oxide nanoparticles when induced by a weak magnetic field.

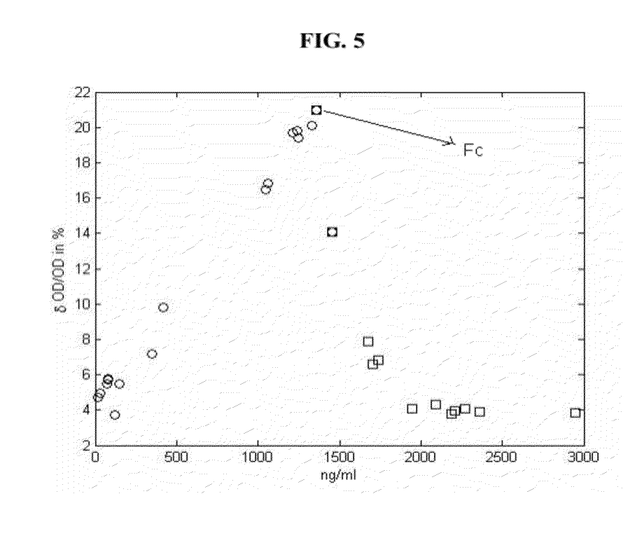

To study how such change in absorbance depends on the estimated ferritin level serum samples with different ferritin concentrations were taken (see FIG. 5). The % decrease in absorbance at 280 nm shows a prominent criticality. When the ferritin level reaches the range 1400-1500 ng/ml, the magnetic effect was drastically changed. The decrease in absorbance however remains almost linear until the critical level of ferritin, F_{c} (see F_{c} in FIG. 5). Beyond F_{c} the % OD decreases with increasing level of ferritin (see the square points mostly taken from thalassemia patients showing iron overloading).

This anomaly was unexpected. By reconstitution study it has already been observed that in the presence of excess iron (here ferritin), there was a formation of anti-ferromagnetically coupled clusters in almost half of total iron content. These clusters of iron do not show any attractive interaction with the magnetic nanoparticle and remain in the solution. The observation presented here is an indication that such ferritin dependent shift of magnetic properties may be a component of iron regulation; a lower ferritin level maintains the magnetic properties per unit ferritin constant, with magnetic properties per unit ferritin level decreasing once such critical ferritin level is reached. Thus, the ferritin assay may find a number of uses in the field of clinical diagnostics related with iron disorders.

### Example 2

### SPIONs as a Ferritin Biosensor

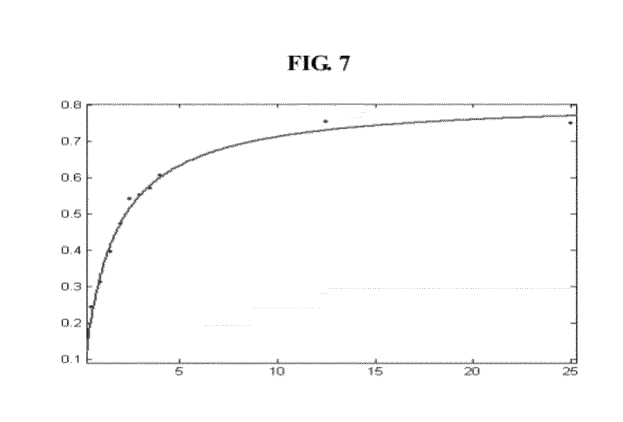

In this example, the peroxidase activity of SPIONs was used to detect serum ferritin. SPIONS function as an artificial inorganic peroxidase. Peroxidases release oxygen and water from hydrogen peroxide enzymatically. SPIONS show similar behavior with Km and Vmax dependent on the SPION surface. Peroxidase activity was found to follow Michaelis Menten kinetics. The peroxidase activity of synthesized SPIONs was examined using TMB as substrate at room temperature. Peroxidase activity of SPIONs (Michealis-Menten Kinetics) was measured using TMB as substrate (Kₘ=0.8139 mM and Vₘₐₓ=4.8*e−7 Abs at 652 nm/sec). Data were fit to the Michaelis-Menten equation (curves shown in FIG. 7, abscissa and ordinate representing time (sec) and absorbance at 652 nm, respectively).

Next, we used the peroxidase activity in measurement of ferritin. This was performed to check errors arising out of contribution of metallic absorption at 280 nm (this may interfere with the protein (trp) absorbance. The peroxidase activity also served as a measure of ferritin specificity of SPIONS. The result are summarized in Table 1. While the ferritin binding shows a linear dependence to 1200 ng/ml, the percent change in activity (absorbance at 652 n m) decreased beyond this value indicating critical magnetic property change beyond the said ferritin concentration. The BSA showed a basal level binding that was not correlated to the BSA concentration. The differential behavior with magnetic incubation may arise because of differential BSA coating of the SPION. In serum, the BSA level is likely to remain of the same order and variation of ferritin is likely to be well reflected by the interaction of ferritin with SPIONs.

TABLE 2
Peroxidase Activity of SPIONs (H == Magnetic Field 20Kgauss)

| | | | | | | |
|---|---|---|---|---|---|---|
| Peroxidase | 0.695 | 0.602 | 0.644 | 0.578 | 400 | 564.5 |
| Activity | 0.695 | 0.567 | 0.565 | 0.577 | 800 | 1129 |
| (Abs at | 0.629 | 0.453 | 0.697 | 0.645 | 1200 | 1693.6 |
| 652 nm) | 0.691 | 0.664 | 0.695 | 0.644 | 2000 | 2822.7 |
| | H− | H+ | H− | H+ | ferr_conc. | BSA_conc |
| | absence | presence | absence | presence | (ng/ml) | (ng/ml) |

| | | |
|---|---|---|
| SPION+ | pure_ferritin | BSA |

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for measuring the level of ferritin in a sample from a subject, the method comprising:
contacting the sample with one or more magnetic nanoparticles;
measuring an absorbance of the sample in the presence and in the absence of a magnetic field; and
comparing the absorbance of the sample in the presence of the magnetic field to the absorbance of the sample in the absence of the magnetic field to determine the level of ferritin in the sample.

2. The method of claim 1 further comprising comparing the measured level of ferritin in the sample to a reference level, wherein a difference in the level of ferritin in the sample compared to a reference level indicates a diagnosis of an iron-related disorder in the subject.

3. The method of claim 1 further comprising comparing the measured level of ferritin in the sample to a reference level, wherein a measured level of ferritin in the sample that is substantially the same as the reference level indicates the absence of an iron-related disorder in the subject.

4. The method of claim 1, wherein the one or more magnetic nanoparticles are superparamagnetic iron oxide nanoparticles (SPIONs).

5. The method of claim 4, wherein the SPIONs are functionalized SPIONs.

6. The method of claim 5, wherein the functionalized SPIONs are functionalized with citrate.

7. The method of claim 4, wherein the SPIONs have a polydispersity from about 0.25 to about 0.29

8. The method of claim 4, wherein the SPIONs have a mean diameter from about 18 nm to about 22 nm.

9. The method of claim 1, wherein the absorbance is measured at about 280 nm.

10. The method of claim 1, wherein the sample is a body fluid sample.

11. The method of claim 10, wherein the body fluid sample is a serum sample.

12. An in vivo method for measuring the level of ferritin in a subject, the method comprising:
administering to the subject an effective amount of one or more magnetic nanoparticles;
measuring one or more physical characteristics of the subject in the presence of a magnetic field; and
comparing the one or more physical characteristics in the presence of the magnetic field to a reference level to determine the level of ferritin in the subject.

13. The method of claim 12, wherein the one or more physical characteristics include a relaxivity R2 measured by MRI.

14. The method of claim 12, wherein the one or more physical characteristics are the optical properties of a body region.

15. The method of claim 14, wherein the optical properties of a body region include the optical absorbance, emission, or scattering of the body region.
